(19) United States
(12) Patent Application Publication
(10) Pub. No.: US 2024/0081699 A1
(43) Pub. Date: Mar. 14, 2024

(54) WIRELESS TISSUE OXYGENATION MONITORING DEVICE

(71) Applicant: VYDAR MEDICAL, Folsom, CA (US)

(72) Inventors: Barbara Sue FARIES, Folsom, CA (US); Wayne T. PAN, Oakland, CA (US); Rajagopal SRINIVASAN, Baltimore, MD (US)

(21) Appl. No.: 18/517,061

(22) Filed: Nov. 22, 2023

Related U.S. Application Data
(63) Continuation of application No. PCT/US22/30599, filed on May 23, 2022.
(60) Provisional application No. 63/192,064, filed on May 23, 2021.

(51) Int. Cl. A61B 5/1455; A61B 5/00

(52) U.S. Cl. CPC A61B 5/14552; A61B 5/0015; A61B 5/6831; A61B 5/6832; A61B 5/7271; A61B 5/742; A61B 5/746; A61B 2560/045; A61B 2560/0462; A61B 2562/0238; A61B 2562/146; A61B 2562/16

(57) ABSTRACT

A tissue oximeter is provided including a wearable sensor unit including a skin contact detector having at least one electrode configured to provide a detection signal when a contact surface is in contact with the skin of a subject; at least one tunable light source arranged to provide a first and second beam of light at two wavelengths; a photodetector arranged to receive the first beam of light and the second beam of light that are reflect from the tissue of a subject.

### CROSS-REFERENCE TO RELATED APPLICATIONS

The present application is a U.S. continuation patent application of, and claims priority under 35 U.S.C. § 120 to, International Application No. PCT/US2022/030599, filed May 23, 2022, designating the U.S., and entitled “WIRELESS TISSUE OXYGENATION MONITORING DEVICE,” which '599 application published as WO 2022/251139 A1 on Dec. 1, 2022, which each of the '599 application and the application publication thereof is incorporated by reference in its entirety herein, and which '599 application, for purposes of the United States, is a U.S. nonprovisional patent application of, and claims priority under 35 U.S.C. § 119(e) to, U.S. provisional patent application Ser. No. 63/192,064, filed May 23, 2021, which '064 application is incorporated by reference in its entirety herein.

### BACKGROUND OF THE INVENTION

### 1. Field of Invention

The present invention generally relates to optical systems that contemporaneously monitor vascular, hemodynamic, and metabolic parameters, including oxygen levels in tissue and blood. Specifically, the invention relates to a miniaturized, wireless, optical tissue and blood oximeter. The device measures oxygenation variation while monitoring optimal microvascular oxygen exchange in sepsis, shock, and other monitored conditions.

### 2. Description of the Related Art

Near-infrared spectroscopy has been used for non-invasive measurement of various physiological properties in animal and human subjects. The basic principle underlying near-infrared spectroscopy is that biological tissues contain various chromophores in a mileu that is relatively transparent to the near-infrared waves. Human tissue contains numerous chromophores such as oxygenated hemoglobin, deoxygenated hemoglobin, water, lipid, and cytochrome. Hemoglobin is the dominant chromophore in the near-infrared spectral range of approximately 700 nm to approximately 900 nm. The near-infrared spectroscope measures differential light transmission or reflection at various wavelengths by the chromophores in the tissue to estimate, for instance, the oxygen saturation of hemoglobin within the tissue.

Time-resolved spectroscopy (TRS), phase modulation spectroscopy (PMS), and continuous-wave spectroscopy (CWS) are some of the various techniques using near-infrared spectroscopy. By solving a photon diffusion equation to calculate oxygenated and deoxygenated hemoglobin concentration and tissue oxygen saturation. TRS and PMS, obtain spectra of an absorption coefficient and reduced scattering coefficient of the physiological medium in both a homogeneous and semi-infinite model. Generally, the design of CWS solves a modified Beer-Lambert equation and measures changes in oxygenated and deoxygenated hemoglobin concentrations.

Despite their capability to provide the hemoglobin concentrations and oxygen saturation, one major drawback of TRS and PMS is that the equipment is bulky and expensive. Devices for CWS may be manufactured less expensively, but CWS is limited in its usefulness because it cannot compute the oxygen saturation changes in oxygenated and deoxygenated hemoglobin concentrations.

Optical Diffusion Imaging and Spectroscopy (ODIS, also known in some applications as Diffuse Optical Spectroscopic Imaging or DOSI) allows tissue to be characterized based on photon scattering and absorption measurements. In tissue such as human tissue, near-infrared light is highly scattered and minimally absorbed. Optical diffusion imaging is achieved by sending optical signals into tissue and measuring the corresponding diffuse reflectance or transmittance on the tissue surface.

Therefore, the heterogeneous tissue structure causes scattering and indicates the cell's density and nuclear size. Interaction with chromophores causes absorption. ODIS emits light into the tissue through a sensor. The light source position, which emits the light, and a detector that detects the light, allow a determined measurement depth. Using a ratio of oxyhemoglobin and deoxyhemoglobin allows for real-time measurement of oxygen saturation levels.

Hemodynamic monitoring and management of patients with sepsis or shock routinely focus on resuscitation and optimization of macro-hemodynamics, such as blood pressure, heart rate, and pulse oximetry. These gross macroscopic assessments may miss the initial subtle manifestation of sepsis and certain shock states. Due to inherent compensatory mechanisms, macroscopic parameters may only show changes after the compensatory mechanisms fail in maintaining homeostasis. A goal of resuscitation and management of sepsis and shock patients is to ensure oxygen delivery, creating optimal microvascular conditions to the vital organs and tissues. Microcirculation is a vital component of the cardiovascular response as it facilitates gas and nutrient exchange at the tissue level. Integrating tissue oximetry into the resuscitative algorithm and coordinating unique monitoring guardrails yield additional physiologic and clinical insights to guide numerous resuscitation aspects.

The assessment of the adequacy of oxygen delivery has traditionally involved invasive measurement of lactate, central (or mixed) venous oxygen saturation (ScvO₂), and global hemodynamic markers, such as mean arterial pressure and cardiac index. The ability to assess oxygen delivery and exchange in a continuous and non-invasive manner significantly increases patient care monitoring and care localization capacity. Pulse oximetry measures macro hemodynamics. However, tissue oximetry can be synergistic with it, as it measures tissue oxygen saturation and reflects a real-time imbalance between oxygen supply and local cellular demand. Also, trending lactates during resuscitation and following lactate clearance in sepsis and shock states have been shown to correlate with outcomes. Tissue oximetry trends during resuscitation correlate with lactate clearance and may represent a non-invasive surrogate for this parameter.

The measurement of oxygen saturation levels in tissue has proved useful in several clinical applications. These applications include resuscitative guidance in hemorrhagic shock, the viability of surgical flaps in reconstructive surgery, cerebral microcirculatory monitoring during cardiac and neurosurgery, athletic training optimization (looking at lactate thresholds), and resuscitative and outcome measures in sepsis and septic shock. This technology also has direct application in equine training optimization, as well as in post-operative and post-chemotherapeutic monitoring within veterinary medicine.

With the COVID-19 global pandemic and the clinical features of this disease process, microcirculatory monitoring related to tissue oxygenation disturbances and hypercoagulable states may prove invaluable. For example, during the current COVID-19 pandemic, patients with serious infection are more likely to develop COVID-19-associated coagulopathy, increasing underlying morbidity and mortality. Usually, the assessment for COVID-19-associated coagulopathy includes monitoring platelet count. PT/aPTT. D-dimer, and fibrinogen levels—but these blood lab tests can only be performed at limited intervals. Recent studies identified an inverse relationship between tissue oxygenation and fibrinolysis (based on D-dimer levels). Non-invasive StO₂ (tissue oxygen saturation) monitoring may be a useful intermediate for early recognition and coagulopathy management by integrating this into the monitoring algorithm.

Additionally, combining these monitoring technologies into a connected, non-invasive, wearable device will enhance the timely clinical approach to therapy guidance in different shock types, as well as in the post-acute, out-of-hospital patient monitoring period. An inexpensive, wireless, miniaturized, non-invasive tissue oximeter that is functionally wearable is needed and advantageous.

### SUMMARY OF THE INVENTION

Patients' respective signs and symptoms in shock or sepsis can be subtle, with common macro-hemodynamic markers commonly delaying presentation or early identification. Despite recent campaigns to drive change, survival rates with septic shock and severe sepsis remain low. While iterations of the systemic inflammatory response syndrome (SIRS) are applied to trigger a cascading intervention response, this response remains based on traditional hemodynamic markers.

Shock and sepsis states have high morbidity and mortality despite current methods of monitoring patient hemodynamics and interventions. This is true for different types of shock that patients may experience including but not limited to septic, cardiogenic, neurogenic, hemorrhagic, and hypovolemic shock. The ability to integrate microhemodynamic measures of perfusion into the current macrohemodynamic pathways through a non-invasive, wireless method with intelligent algorithm associated software alerts will enhance the capacity to provide optimal monitoring and care team response regardless of location. Such a device utilizing multiple wavelength monitoring of oxygen saturation levels in tissue and additional hemodynamic measures within a miniature wireless optical tissue oximeter for sepsis and different types of shock is disclosed.

The present invention relates to a miniaturized wireless, integrated, device that utilizes optical tissue oximetry to monitor tissue oxygenation (StO₂), pulse, surrogate markers for lactate, and potentially a surrogate for coagulopathy levels in sepsis and other types of shock, as well as exercise physiology. This design enables wireless or cloud-based integration into the home. EMS, and hospital-based monitoring systems (and applications). This type of device provides continuous tissue oxygenation readings with integrated trend alarms to foster optimal care management.

The tissue oximeter sensor may be placed on any location of a subject's body where a suitable tissue bed is available. Muscle oximetry requires placement over a well-muscled area, but tissue oximetry may be more varied in location. For example and without limitation, the thenar eminence, deltoid, hamstring, quadriceps, adductor muscles of the thigh, gastrocnemius muscle on the calf, and temporal/lateral forehead regions and other locations may be suitable locations for placing the tissue oximetry device. The tissue oximeter sensor may also be applied in care management for non-human patients. For example, a veterinarian treating animals undergoing chemotherapy treatment or experiencing traumatic injury may use the oximeter device to monitor for shock (septic shock and other types of shock) in his or her animal patients.

In one embodiment, a miniaturized, wireless, optical tissue oximeter is contained within a fixation unit and worn on an individual's hand. The small size makes it easily deployable in pre-hospital/EMS transport, Emergency Department (ED), Intensive Care Unit (ICU), and long term care or at-home monitoring scenarios. The fixation unit adheres to the thenar eminence at the base of the thumb and wraps around to the back of the hand. The portion of the fixation unit located over the thenar eminence contains a sensor connected to a microprocessor powered by a battery or other appropriate energy source.

In another embodiment, the device is attached to other targeted regions, such as the shoulder (deltoid), calf or thigh using the same adherence process. Should the device adherence fixation unit require replacement, such replacement can be accomplished easily by the end user or care provider. In addition, the device may also be wearable utilizing a wrist band, belt, leg band, or clothing clip. Another embodiment may also include a rechargeable alternative for the miniaturized, wireless, wearable device.

In another embodiment used for a fitness application, the microprocessor calculates and transmits an easy-to-use, consumer-friendly exercise index to allow athletes to adjust their exercise intensity level based on the present invention's non-invasive tissue oxygen saturation measurements rather than invasive blood lactate measurements. The exercise index value can be displayed numerically or in the form of an easy-to-understand red, yellow, and green lights in which green indicates the intensity of exercise is in the aerobic range, yellow indicates a transition from an aerobic to an anaerobic state. Red indicates reaching the anaerobic range. Utilizing these parameters will optimize the physiologic training goals desired. This indication can also be applied to non-human athletic training and competition (e.g., equine fitness applications such as racing).

According to an aspect of the present invention, the sensor contains at least one light source that is tunable to emit light at various wavelengths, a photodetector, and a skin contact detector. Once skin contact is detected using the skin contact detector, the light source is tuned to emit light in the near-infrared region (NIR) between 700 and 2500 nm, and the light source is tuned to emit light in the visible red region such as between 620 and 700 nm. In some embodiments, the sensor includes a first light source and a second light source, in which the first light source emits light in the NIR between 700 and 2500 nm, and the second light source emits light in the visible red region such as between 620 and 700 nm.

The emitted light passes through a transparent layer of the fixation unit and enters the underlying tissue. The tissue chromophores, including oxygenated hemoglobin and deoxygenated hemoglobin, absorb a portion of the light, and the remainder is reflected out of the tissue into the photodetector. Oxygenated hemoglobin absorbs more infrared light and deoxygenated hemoglobin absorbs more red light. The sensor calculates the tissue's oxygen saturation as the ratio of the measured concentration of the oxygenated hemoglobin to the measured deoxygenated hemoglobin concentration.

The oxygen saturation measurements may be displayed on a display integrated with the sensor and/or may be wirelessly transmitted to a remote display device, such as a smartwatch, smartphone, or other device running a software application that receives the measurements and displays them in numeric, graphical, and/or audible form. The software application may also relay the data to a patient monitoring system integrated into an external application or health system electronic health record (EHR), web application viewing, or other remote application.

According to another aspect of the invention, the microelectronics and processing module monitors skin contact sensor. Upon detecting skin contact, it automatically increases or decreases the intensity of one or more light sources until the detector receives an adequate signal. This device will sample at multiple wavelengths within the previously described operating range to optimize tissue oxygenation sampling values across the visible red and NIR spectrum. This innovation allows for improved accuracy of the continuous detector measurements minimizing variability. The one or more light sources illuminate sequentially so that a corresponding detector measurement at each wavelength can be uniquely obtained. In some embodiments, the microprocessor contains internal digital to analog converters that control the intensity of the light source. It also includes internal amplifiers and an analog-to-digital converter to obtain measurements from the photodetector.

Furthermore, the electronics, computing, and wireless communications module contains a process, read-only memory, read-write memory, and a serial or other interface to communicate with a wireless transceiver. The module is powered by a miniature battery or other source of electrical energy and may contain internal power conversion circuitry to provide proper power supply voltages to the different submodules.

According to another aspect of the invention, the tissue oxygen saturation measurements may be viewed on a display integral to the device and/or may be wirelessly transmitted to a remote display device utilizing a software application (e.g., integrated medical monitoring device, smartphone, smartwatch, etc.), which receives the measurements and displays them in one or more of numeric, graphical, and audible form. For example, if the sensor is placed on the hand or wrist, a display may be included on a cuff or wristband that holds the sensor in the desired measurement location and the display in an easily viewed position. Sensors placed on other body locations may wirelessly transmit measurements to a remote display carried or worn by a user, such as a smart phone or smart watch. Measurement data may be displayed numerically, graphically and/or visually. In embodiments, the measurements may be converted by algorithms to display an indicator that the tissue oxygenation is in, for example, desirable or undesirable levels or indicators of a level of exertion of the subject. In addition to tissue oxygenation, the display may also display other data such as pulse, temperature, blood pressure, etc. that may be obtained using additional sensors. Data may also be integrated into a cloud-based data analytics platform integrating clinical algorithms that can be pushed for remote viewing on a web site or remote transfer to a hospital patient-monitoring data system.

According to yet another aspect of the invention, the software application calculates and integrates monitoring and trend predictions enabling earlier recognition of pending patient decompensation and possibly medical intervention by tracking changes in StO₂, pulse oximetry, and other hemodynamic measurements over time. These may be quantified in terms of change from baseline (ΔStO₂), changes over time (ΔStO₂/t), and rates of change (ΔStO₂/t*2) This innovation builds in clinical alert algorithms that distill beat-to-beat variability into clinically actionable information. Detecting trends in tissue oxygenation concurrent with patient-specific alert parameters within the algorithm will direct the clinical response. For example, the algorithm monitors the StO₂ values for trend decreases when the values remain in the normal range as well as when the StO₂ is abnormal (below about 70%). A clinical alert will be triggered when there is a continued minute by minute decrease in StO₂ while above the abnormal threshold; a clinical alarm will be triggered when dropping below the abnormal threshold. This provides not just a trend variation alert, but also an absolute alert based on established abnormal monitoring thresholds.

In one aspect of the invention, a portable tissue oximeter is provided including a wearable sensor unit defining a contact surface including a skin contact detector having at least one electrode configured to provide a detection signal when the contact surface is in contact with the skin of a subject; at least one tunable light source arranged to provide a first beam of light at a first wavelength and a second beam of light at a second wavelength; a photodetector arranged to receive the first beam of light and the second beam of light that are reflected from the tissue of a subject. The sensor unit further includes a sensor unit wireless transceiver; and a sensor unit microprocessor and a computer-readable storage medium. The computer-readable storage medium stores instructions that when executed by the sensor unit microprocessor cause the sensor unit microprocessor to initiate intensity of the light source upon receipt of the detection signal; measure the output of the photodetector compute oxygen saturation values based on photodetector measurements; and transfer a signal of the oxygen saturation values from the sensor unit wireless transceiver. The portable tissue oximeter further includes an external device including an external device wireless transceiver; an external device microprocessor and a computer-readable storage medium storing instructions that when executed by the external device microprocessor cause the external device microprocessor to receive oxygen saturation values form the sensor unit transceiver and determine outputs to a user; and a user interface to provide user notifications of oxygen saturation and alerts.

In some embodiments, the at least one tunable light source emits near-infrared light at several different wavelengths within that spectrum. The at least one tunable light source emits visible red light at a wavelength of 660 nm.

In some embodiments, the skin contact detector measures the capacitance of a region of the skin under the detector.

In some embodiments, the sensor unit further includes a fixation system configured to fully contain the sensor unit and secure it to the skin. The fixation system can include a pressure-sensitive adhesive or a strap. In some embodiments, the fixation system includes a biocompatible transparent film to separate the at least one tunable light source, the photodetector, and skin contact detector from the skin. In some embodiments, the fixation system includes a first compartment to house the at least one tunable light source, the photodetector, and the skin contact sensor, wherein the first compartment is oriented to align with the portion of the subject's skin to a be analyzed; a second compartment to house the sensor unit microprocessor, and a third compartment to house the sensor unit wireless transceiver. In some embodiments, the tissue oximeter of claim 5 wherein the fixation system includes a first compartment to house the at least one tunable light source, the photodetector, and the skin contact sensor, wherein the first compartment is oriented to align with the portion of the subject's skin to a be analyzed; and a second compartment to house the sensor unit microprocessor and the sensor unit wireless transceiver. In some embodiments, the fixation system includes a single compartment to house the at least one tunable light source, the photodetector, the skin contact sensor, the sensor unit microprocessor and the sensor unit wireless transceiver.

The user interface includes a display screen to display the oxygen saturation values, alerts, and an indication of skin contact. The sensor unit transceiver and external unit transceiver communicate wirelessly by any applicable protocols such as via cellular communications, Bluetooth. NFC or WiFi.

In some embodiments, the sensor unit microprocessor or the external unit microprocessor is configured to correlate blood lactate measurements with the tissue oxygen saturation measurements. The sensor unit microprocessor or the external unit microprocessor is configured to determine whether the subject is in an aerobic state, in a transition from an aerobic to an anaerobic state, and in an anaerobic state based on the correlation between blood lactate measurements and the tissue oxygen saturation measurements. The user interface provides an alert regarding the state of the subject in an aerobic state, in a transition from an aerobic to an anaerobic state, and in an anaerobic state.

In another aspect of the invention, a portable tissue oximeter is provided including a sensor unit defining a contact surface including a skin contact detector including at least one electrode configured to provide a detection signal when the contact surface is in contact with the skin of a subject; at least one tunable light source arranged to provide a first beam of light at a first wavelength and a second beam of light at a second wavelength; a photodetector arranged to receive the first beam of light and the second beam of light that are reflected from the tissue of a subject; and a microprocessor and a computer-readable storage medium storing instructions that when executed by the microprocessor cause the microprocessor to initiate intensity of the light source upon receipt of the detection signal; measure the output of the photodetector; and compute oxygen saturation values based on photodetector measurements; and a user interface to provide user notifications of oxygen saturation and alerts.

In some embodiments, the at least one tunable light source emits near-infrared light at several different wavelengths within that spectrum. The at least one tunable light source emits visible red light at a wavelength of 660 nm. In some embodiments, the skin contact detector measures the capacitance of a region of the skin under the detector.

In some embodiments, the sensor unit further including a fixation system configured to fully contain the sensor unit and secure it to the skin. The fixation system can include a pressure-sensitive adhesive or a strap. The fixation system includes a biocompatible transparent film to separate the at least one tunable light source, the photodetector, and skin contact detector from the skin. In some embodiments, the fixation system includes a first compartment to house the at least one tunable light source, the photodetector, and the skin contact sensor, wherein the first compartment is oriented to align with the portion of the subject's skin to a be analyzed; and a second compartment to house the microprocessor. In some embodiments, the fixation system includes a single compartment to house the at least one tunable light source, the photodetector, the skin contact sensor, and the microprocessor.

The user interface includes a display screen to display the oxygen saturation values, alerts, and an indication of skin contact. In some embodiments, the microprocessor is configured to correlate blood lactate measurements with the tissue oxygen saturation measurements. The microprocessor is configured to determine whether the subject is in an aerobic state, in a transition from an aerobic to an anaerobic state, and in an anaerobic state based on the correlation between blood lactate measurements and the tissue oxygen saturation measurements. The user interface provides an alert regarding the state of the subject in an aerobic state, in a transition from an aerobic to an anaerobic state, and in an anaerobic state.

These and other advantages of the present invention become apparent by reading the following detailed descriptions, figures, and diagrams.

### BRIEF DESCRIPTION OF THE DRAWINGS

FIGS. 1A and 1B show aspects of the difference between pulse oximetry and tissue oximetry in accordance with an embodiment of the present invention.

FIG. 2A is a block diagram representation of a wireless monitoring device for sepsis and shock in accordance with an embodiment of the present invention.

FIG. 2B is a side view of the wireless monitoring device of FIG. 2A in accordance with an embodiment of the present invention.

FIG. 3A is a diagrammatic top view representation of a wireless monitoring device for sepsis and shock in accordance with an embodiment of the present invention.

FIG. 3B is a diagrammatic side view representation of a wireless monitoring device for sepsis and shock in accordance with an embodiment of the present invention.

FIG. 4A is a perspective view of a wireless monitoring device for sepsis and shock placed on a hand.

FIG. 4B is a perspective view of a wireless monitoring device for athletic training applications placed on an athlete's gastrocnemius (or deltoid).]

FIG. 4C is a block diagram representation of an external device for connecting wirelessly with the sensor unit in accordance with an embodiment of the present invention.

FIG. 5 is the device process flowchart in accordance with an embodiment of the present invention for sepsis and shock applications incorporating software analysis and clinical alert processes.

FIG. 6 is a time-course plot of tissue oxygen saturation obtained from an embodiment of the present invention.

FIG. 6A is a sample clinical process map in accordance with an embodiment of the present invention for sepsis and shock applications (including COVID-19).

FIG. 7 is a device process flowchart in accordance with an embodiment of the present invention for athletic training and fitness applications.

FIG. 8 is a plot of blood lactate as a function of running speed before and after physical training in accordance with an embodiment of the present invention.

FIG. 9 is a plot of breakpoint workload derived from tissue oxygen saturation versus breakpoint workload derived from lactate in accordance with an embodiment of the present invention.

FIGS. 10A through 10E are examples of exercise index versus exercise stage graphs in accordance with an embodiment of the present invention.

FIGS. 11A through 11E are examples of exercise index versus lactate graphs in accordance with an embodiment of the present invention.

### DETAILED DESCRIPTION OF THE PREFERRED EMBODIMENTS

The present invention relates to a miniature, wireless device that detects tissue oxygenation. In some embodiments the device also integrates additional hemodynamic and/or physiologic monitoring such as pulse, pulse oximetry and hemoglobin levels for sepsis, shock, and exercise training applications. The oximeter measures local tissue oxygen saturation (StO₂) using red and near-infrared spectroscopy. In embodiments, the device also measures pulse oximetry. The measurement is non-invasive, immediate, and continuous. The device may be used for human and certain animal subjects in veterinary applications.

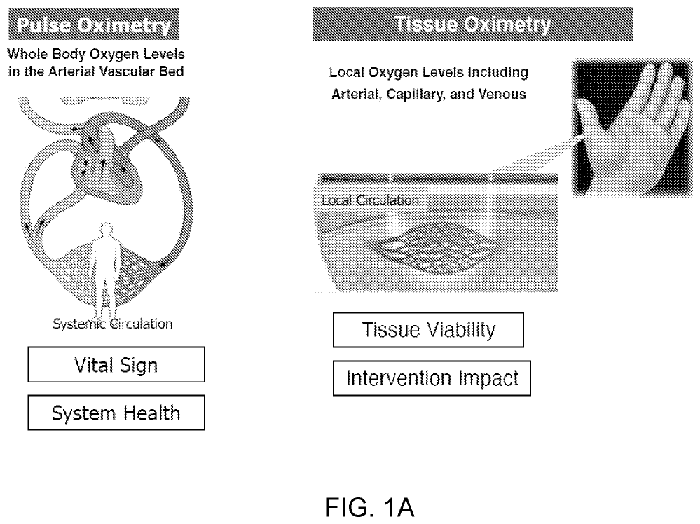

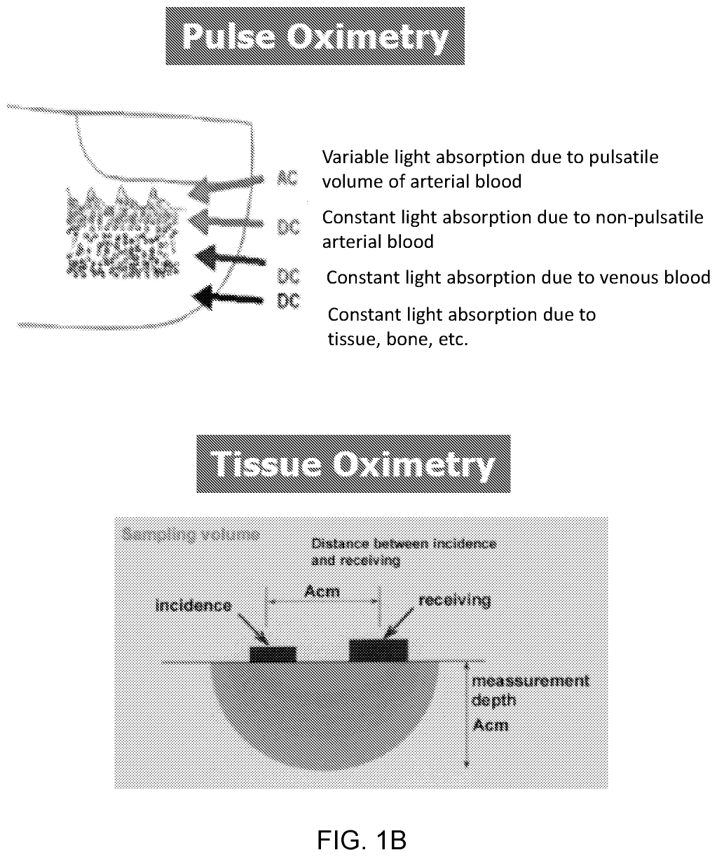

As shown in FIGS. 1A and 1B, pulse oximeters and tissue oximeters measure different parameters, and have different applications. Pulse oximeters measure the whole body oxygen saturation of hemoglobin located in the arterial blood only. This corresponds to the small change in signal that occurs due to the pulse, which is all arterial blood in content. It is a measure of how well the lungs are oxygenating the arteries systemically. Pulse oximeters rely on the difference between pulsatile, arterial blood signals and non-pulsatile, non-arterial blood and other tissue attenuation. In the absence of a pulse, no measurement can be obtained. Measurement is based on light reflected from the sample. As used herein, it is understood that depending upon the tissue configuration, light “reflected” from the sample can mean that light is transmitted or scattered from the sample.

Tissue oximeters measure the oxygen saturation of hemoglobin located locally in a volume of tissue under the sensor, which is a combination of arterial, capillary, and venous blood. No pulse is required, and a sensor can be placed anywhere on the body, such as upper or lower limbs, including, for example, the calf, thigh, shoulder, upper arm, wrist, or hand. It is an indicator of tissue perfusion and can be used to determine the impact (negative or positive) of an intervention on the subject. Tissue oximeters rely on the reflected light properties detected within any tissue. No pulse is required, and measurement is based on light reflected from the sample. This distinction is significant in various shock states when peripheral vasoconstriction limits pulse oximetry detection quality.

In some embodiments, the tissue oximeter device includes a sensor unit and an external device. In some embodiment, the sensor unit is integral with a display unit and is a single unitary device.

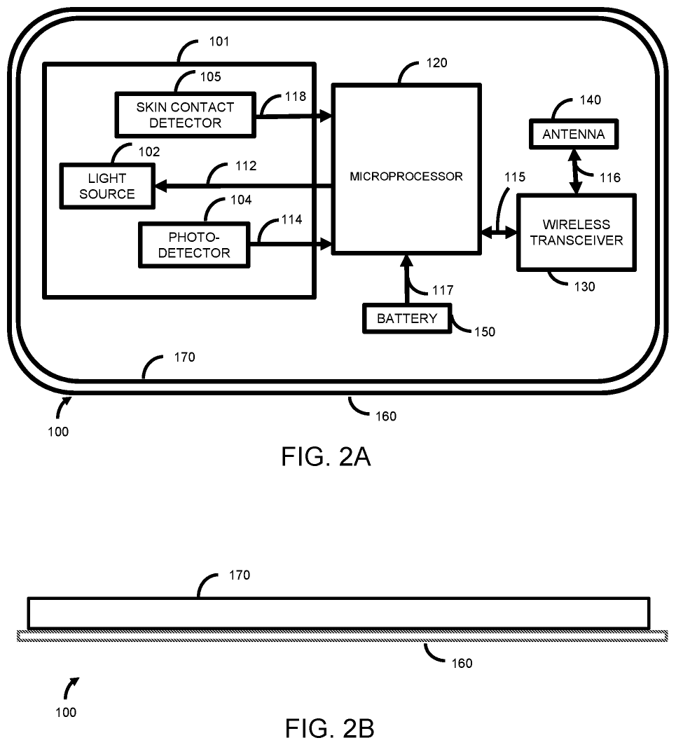

FIG. 2A is a block diagram representation of a wearable sensor unit 100 for use with an external device 500. The sensor unit 100 is a “wearable” device that can be secured to the skin of the subject and worn comfortably for an extended period of time with functionality. As a wearable device, sensor unit 100 is secured to the subject and provides tissue oxygenation values to an external device 500 via a wireless protocol. FIG. 2B illustrates a side view of the sensor unit 100 including a fixation system having a housing 170 and adhesive strip 160. In some embodiments, housing 170 has dimensions of about 4 cm×3 cm×0.5 cm with a volume of about 6 mL. In some embodiments, the volume of the housing can be about 5 mL, 6 mL, 7 mL, 8 mL. 9 mL, 10 mL, 15 ml, or 20 mL.

Sensor unit 100 includes a multiple wavelength optical sensor portion 101, which contains a light source 102, a photodetector 104, and a skin contact detector 105. In some embodiments, wearable sensor unit 100 includes one light source capable of tuning to plurality of wavelengths and intensities. In some embodiments, wearable sensor unit 100 includes two or more light sources. The light source 102 can be configured to emit a first beam of light in the near-infrared region into the tissue, and configured to emit a second beam of light in the visible red region into the tissue. By way of example, the light source is tuned to emit the first beam of light at variable wavelengths within the near-infrared region (700-900 nm) and configured to emit light a second beam of light at a wavelength of about 660 nm in the visible electromagnetic spectrum. The selected wavelengths and utilization of several different wavelengths optimize the sampling between oxy- and deoxy-hemoglobin. However, the wavelengths of light produced by light-emitting diodes associated with light source 102 may vary widely, thus allowing for a broad range of oximetry wavelength data capture. The wavelength and intensity of light emitted by the light source 102 can be controlled by microprocessor 120 via connection 112.

The first beam of light and the second beam of light enter the tissue, with a portion of each beam reflected by the tissue and received by the photodetector 104. Photodetector 104 may detect reflected wavelengths broadly, and the reflectance spectrum is analyzed near 900, 760, 700 and 600 nm to determine hemoglobin saturation levels.

In some embodiments, the light source 102 and the photodetector 104 are tunable by the microprocessor to emit and detect light, respectively, at different selected wavelengths. For example, when a light beam emitted by light source 102 is tuned to a particular wavelength by microprocessor 120, the photodetector 104 is likewise tuned by microprocessor 120, e.g., by use of gratings or filters, to selectively detect the level of reflected light at the selected wavelength. Notably, the light source 102 and the detector 104 are disposed in the sensor portion 101 so that the detector 104 does not detect any light directly emitted by the light source and can detect only light reflected by the tissue. In some embodiments, these components may form a fully self-contained single miniature monitoring device. In still further embodiments, the monitoring device may be disposable.

In addition, sensor portion 101 include a skin contact detector 105. The skin contact detector 105 can be located proximal to the light 102 and photodetector 104 to detect contact with the skin. In some embodiments, skin contact detector 105 include a single contact patch or electrode. In some embodiments, the skin contact detector 105 includes a plurality of contact patches or electrodes. Multiple contact patches or electrodes can monitor the entire critical surface as a matrix configuration. The skin contact sensor utilizes capacitive, resistive and/or inductive sensors to indicate that the sensor is in contact with the skin. For example, skin contact detector 105 may consist of a planar conductive element forming the first plate of a capacitor, adjacent to one or more conductive elements forming the second plate of a capacitor. The skin contact detector 105 is electrically insulated from the skin using the adhesive strip 160. The total capacitance value between the first plate and the second plate increases by contact with human skin, which serves as an electrical dielectric. Therefore, measurement of the capacitive value allows for the detection of skin contact wherein a threshold level of capacitance indicates sufficient skin contact. The threshold value is within the lower human body capacitance range of 100-200 pF.

The sensor portion 101 interconnects with a microprocessor 120. These connections comprise connection 112 joining the light source 102 to microprocessor 120 through which the microprocessor 120 can control the intensity of the light source 102, connection 114 joining the photodetector 104 to microprocessor 120 through which the microprocessor 120 can measure the electrical signal from photodetector 104, and connection 118 joining skin contact detector 105 to microprocessor 120 through which the microprocessor can detect whether the sensor portion 101 is in contact with skin.

The microprocessor 120 receives power from an energy source such as traditional electrochemical DC cell battery 150 using connection 117. In embodiments, the battery 150 may have a lifetime of up to two weeks, or longer and may be rechargeable in some embodiments. In other embodiments, supercapacitors may be used. The microprocessor 120 can include internal power conversion circuitry to provide supply voltages to the wireless transceiver 130

The microprocessor 120 contains internal digital-to-analog converters controlling the intensity of the light source 102 and includes internal amplifiers and an analog-to-digital converter to obtain measurements from the photodetector 104. Furthermore, the microprocessor 120 further contains storage including read-only memory, read-write memory for storing instructions to operate the wearable sensor unit 100. The microprocessor 120 also includes a serial or other interface to communicate with wireless transceiver 130. The microprocessor enables integration of algorithmic and intuitive data processing at the source, furthering future device applications. An example of a microprocessor is a programmable system-on-a-chip such as, e.g., Cypress Semiconductor PSoC® 6 CY8C62×6. An example of a wireless transceiver with a connected antenna is the Roving Networks RN4870 Bluetooth Module. As future iterations or other wireless modalities develop, these technologies may be integrated in different ways, and other microprocessor and wireless transceiver products may be used successfully in the wearable sensor unit 100.

An optional adhesive strip 160 may be attached to some or all the system components, including sensor portion 101, microprocessor 120, battery 150, wireless transceiver 130, antenna 140, and associated interconnections, thereby forming a fully self-contained miniature monitoring device. In embodiments, the components are attached to the adhesive unit 160 using a double-sided adhesive. Adhesive strip 160 is shown diagrammatically as a unitary piece, but that is not limiting. In embodiments, the adhesive strip 160 may be divided into a plurality of portions wherein one or more of the components of the device are on separate portions to provide more flexibility of placement of the device on a subject's skin. For example, sensor portion 101 may be attached to a first region of the adhesive strip 160 and components 120, 150, 130, 140 are on a second separate region of the adhesive strip 160 and connected to the sensor portion 101 with electrical leads. The adhesive strip 160 may include portions near the sensor portion 101 that allows light to pass through at the wavelengths emitted by light source 102.

In embodiments, instead of an adhesive strip 160, the device may comprise a generally wafer- or disc-shaped casing 170 to contain the components of the device, thereby forming a fully self-contained miniature monitoring device. The casing generally comprises a skin contact face disposed in front of the sensor portion 101 that allows light to pass through the casing via specific routes and optical windows. For example, the case material may be transparent to light emitted by the light source 102 and received by the photodetector 104. The other surfaces of the casing 170 are preferably opaque to prevent ambient light from reaching photodetector 104. In some embodiments, the microprocessor 120, transceiver 130, antenna 140 and battery 150 are disposed within the casing behind the sensor portion 101.

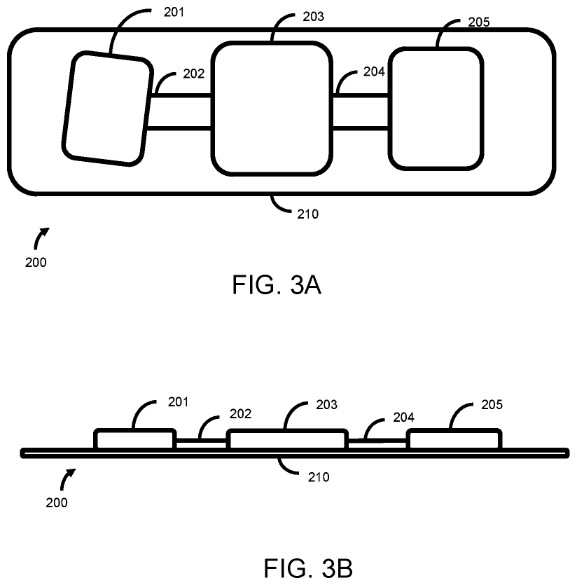

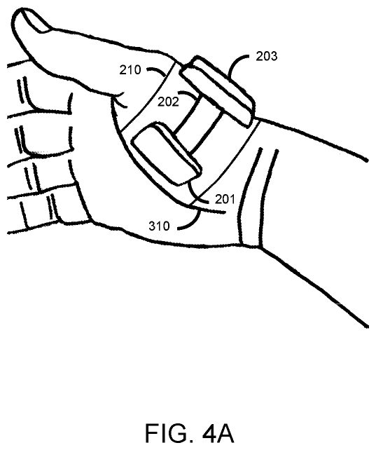

In some embodiments, the adhesive strip 160 is non-continuous, such as about the area of each of the compartments, described in relation to FIGS. 3A and 3B, the components could be placed on the hand in different positions depending on the subject's hand size.

The adhesive strip 160 is constructed of a wearable, gauze, woven textile, or similar wrap knit or stretchable, wear-safe material with a pressure-sensitive adhesive (PSA) on the skin contact surface. Notably, the portion of the adhesive strip 160 proximate to the sensor portion 101 is transparent to light of the relevant wavelengths in order to allow light from the light sources to illuminate the skin and allow reflected or scattered light to reach the detector. The skin contact surface will include a non-irritating bio-adhesive, such as a hydrocolloid, to facilitate extended wear up to several weeks, despite body movement. In embodiments, only the sensor portion 101 needs to be attached to the subject's skin and the other components may be disposed on a substrate that can be wrapped around a portion of the subject's body to be held in place. For example, when the device is configured to measure tissue oximetry at a subject's thenar eminence, the sensor portion 101 may be attached to the thenar eminence and the other components disposed on a wristband or cuff that is not adhesively attached to the subject. In some embodiments, the adhesive strip 160 may be attached to the subject's skin and then one or more of the components of the device attached to the adhesive strip 160 to allow for later removal and reuse.

In some embodiments, microprocessor 120 transmits some or all measurements and computed values to the wireless transceiver 130 via connection 115, which transmits and receives information from antenna 140 using connection 116.

FIG. 3A is a diagrammatic top view representation of a sensor unit 200 in accordance with an embodiment of the present invention. Sensor unit 200 can be used with an external device 500. The components referred to FIG. 2, including the sensor portion 101, including the light source 102, photodetector 104, skin contact detection 105, microprocessor 120, power source 150, and wireless transceiver 139 are also incorporated into sensor unit 200.

A fixation system is provided to contain the sensor portion 101 and secure it to the skin of the subject. In some embodiments, a fixation system includes one or more opaque compartments 201/203/205, flexible connectors 202/204 and an adhesive film 210. The sensor unit 200, including sensor portion 101, compartments 201/203/205, flexible connectors 202/204 and adhesive film 210, is a “wearable” device that can be secured to the skin of the subject and worn comfortably for an extended period of time with functionality. As a wearable device, sensor unit 200 is secured to the subject, and provides tissue oxygenation values to the external device 500 via a wireless protocol. In some embodiments, the over

Opaque compartment 201 houses sensor portion 101 and secures it to biocompatible transparent PSA film 210. Notably, a portion of compartment 201 that contacts the subject's skin is transparent to select wavelengths allow light to pass through. Compartment 203 houses the microprocessor 120 and battery 150, securing them to the transparent PSA film 210. Compartment 205 houses the wireless transceiver 130 and antenna 140 and secures them to the transparent PSA film 210. Compartment 201 is electrically connected to compartment 203 using a flexible connection 202. Compartment 203 is electrically connected to compartment 205 using a flexible connection 204. The flexible fixation unit includes the body contact surface, materials (adhesives) for securing compartments and an outer protective layer. There are numerous examples of transparent non-latex PSA films available commercially from companies such as DuPont. 3M and Scapa. Specific examples of film 210 include Scapa RX1402P, a biocompatible 0.003 inch-thick polyethylene film single coated with a PSA, Liveo (Dupont) and 3M medical grade adhesives. An example of a material for compartment 201, compartment 203, and compartment 205 is Scapa 0399003, a non-latex, biocompatible ⅛ inch-thick polyethylene foam single-coated with a PSA, covered by an outer opaque layer of Scapa RX848P biocompatible metalized polypropylene film. A removable cover layer for protecting the PSA until the device is to be applied to a subject's skin may also be used but is not shown in the Figures.

In some embodiments, the components of the device can be placed between the film and foam layers and the layers laminated together using known lamination procedures. For example, a base layer comprising film 210 (and cover layer) is fed to a horizontal continuous lamination machine, and the components are placed thereon using a conveyor system. The foam layer and cover layers can be fed to the laminator to overlay the components and then laminated to the base layer film 210 using heat and/or pressure to form compartments 201, 203 and 205. The laminated films can be cut apart to provide individual devices 200.

In embodiments, film 210 comprises a PSA coating on a first side configured to be attached to compartments 201, 203 and/or 205 and a biocompatible skin contact PSA configured to adhere to the subject's skin. After use, the adhesive film 210 can be removed from the skin allowing the device to be removed from the film 210 for reuse.

FIG. 3B is a diagrammatic side view representation of the sensor unit 200 in accordance with an embodiment of the present invention. The emitted light from light source 102 within sensor compartment 201 passes through transparent PSA film 210 of the adhesive fixation unit. It enters the tissue, to which transparent PSA film 210 has been applied, where tissue chromophores absorb a portion of the light, including oxygenated hemoglobin and deoxygenated hemoglobin, and a portion of the non-absorbed light is reflected out of the tissue to photodetector 104 contained within compartment 201. Those familiar with the art will recognize that the relative light levels detected at different wavelengths can be correlated with hemoglobin saturation levels measured using invasive blood analysis and laboratory techniques. The amount of reflected light is a portion of the emitted light that is dependent on the amount of light absorbed by chromophores such as oxygenated hemoglobin and deoxygenated hemoglobin and is related to the concentration of these chromophores in the tissue. The oxygen saturation of the tissue under the sensor is then estimated as the ratio of oxygenated hemoglobin to total (oxygenated plus deoxygenated) hemoglobin. To validate the device, the level of hemoglobin determined using methods described herein from reflected light at the indicated wavelengths has been correlated to hemoglobin amounts determined by blood analysis.

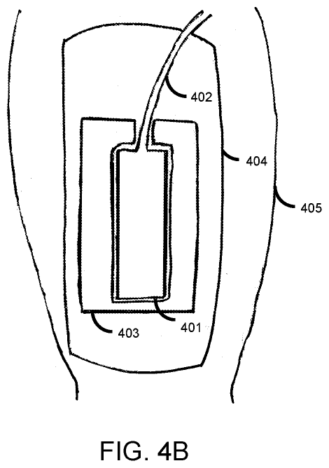

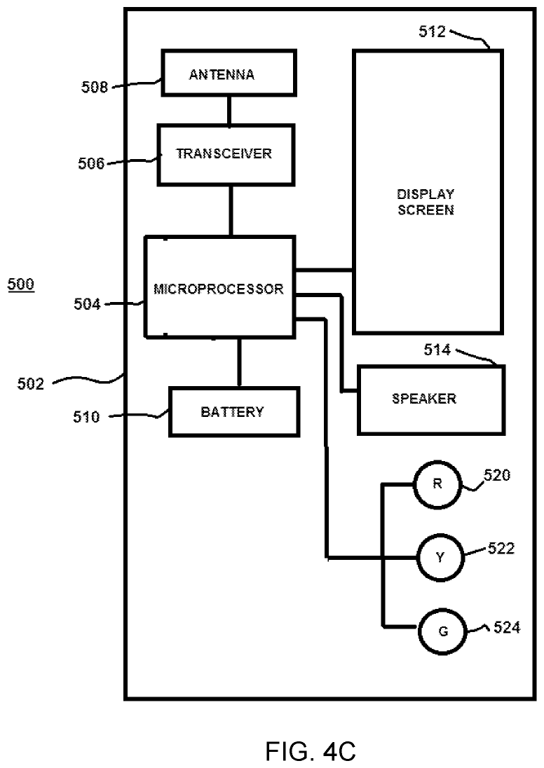

FIG. 4A is a perspective view of a sensor unit 200 placed on and secured to a hand. Transparent, flexible, PSA film 210 is positioned and secured with the adhesive to the hand so that the long axis of sensor compartment 201 aligns with the long axis of the thenar eminence 310 of the hand. Compartment 203, containing the microprocessor and battery, is positioned over the back of the hand's dorsal aspect. Compartment 201 is electrically connected to compartment 203 using flexible connection 202. Not shown in the figure, the PSA film wraps around to the back of the hand and secures compartment 205 thereto.

In other embodiments, the components of the device can be held in place using an elastic band, strap or cuff such that the sensor compartment 201 is disposed on the thenar eminence, optionally with a skin adhesive film, and the other components disposed elsewhere on the elastic band, strap or cuff. In some embodiments, the

FIG. 4B is a perspective view of another embodiment of the present invention, in which reusable sensor unit 401 containing sensor portion 101 is contained within but removable from compartment 403, which may comprise a biomedical foam layer and cover layer such as described above for device 200. Compartment 403 is attached to a flexible, transparent PSA film 404, applied with biocompatible PSA to the skin of a subject. In this figure, the device is attached to the calf 405 of a subject, but other locations are envisioned depending on where tissue oximetry is needed, such as shoulder, thigh, back, temporal or forehead regions of the subject, Reusable sensor element 401 is electrically connected to the microprocessor 120 using electrical cable 402. Microprocessor 120 is in turn connected to battery 150, wireless transceiver 130 and antenna 140. Compartment 403 and the flexible, transparent PSA film 404 forms a replaceable fixation unit, which can be removed from the subject and the device components. In other embodiments, sensor unit 401 may be held in place with a non-adhesive fixation unit, such as a sleeve or band configured to wrap around the subject's calf.

FIG. 4C illustrates an external device 500 for use with devices 200 or 401. External device includes a housing 502 constructed of a lightweight material such as plastic to house the components of the device. In some embodiments, external device can take on the dimensions of a watch housing, or a mobile phone. External device can be a single use dedicated unit for tissue oximetry, or external device can be a mobile phone or tablet. Housing can further include a wrist strap or clip (not shown). A microprocessor 504, in connection with storage, executes operations to provide output of tissue oximetry and other information. A power supply, such as a DC battery 510 powers the device 500. The oximetry readings from device 200 are sent to device 500 via wireless protocol such as cellular, WiFi, Bluetooth or NFC and received at transceiver 506 via the antenna 508. Output devices include a display screen 512, such an LCD or OLED display can provide information such tissue oximetry readings and time plots, indications that skin contact has occurred, indication of connectivity with device 200, as well as alerts. A speaker 514 provides alerts by the use of tones. A series of lights, e.g., red 520, yellow 522, and green 524 are visible on the housing body 502.

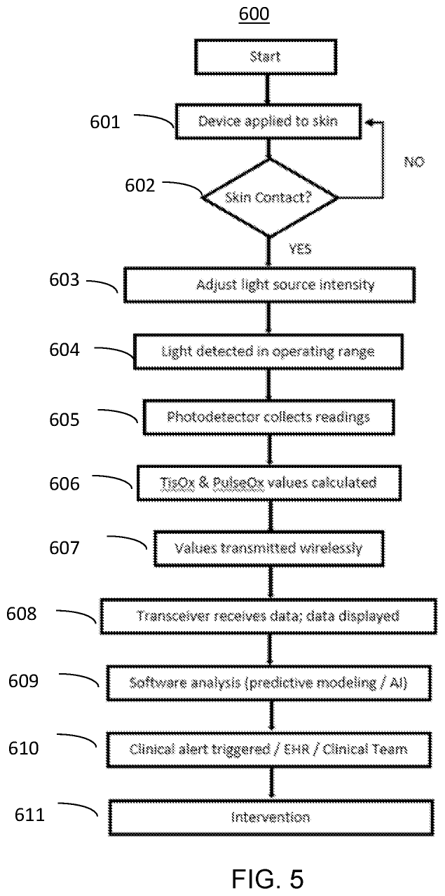

FIG. 5 is a process flow diagram in accordance with an embodiment of the present invention, illustrating one method by which the microprocessor 120 can obtain and wirelessly transmit oxygen saturation values. The process 600 begins with application of the sensor unit 100/200 to the skin in step 601. The one or more electrodes of the skin contact detector 105 provide a signal representative of contact with the skin. For example, if skin contact is detected, a display icon is shown on a display 512 of a personal and/or monitoring center associated with the external device 500, an audible signal and/or a haptic output is provided to indicate that skin contact has been achieved. The absence of such indication can indicate a lack of skin contact. If skin contact is not detected at decision block 602, e.g., no signal is provided by skin contact detector 105, device re-application is needed (return to Step 601) until skin contact is detected. Confirmation of connectivity of the transceiver 130 with an external device by the microprocessor 120 can be achieved by acknowledgement of reception of measurements obtained from skin contact detector 105. Other techniques may be used to confirm connectivity between the transceiver 130 and the external device.

Once skin contact is detected, the light intensities from light source 102 are automatically varied by microprocessor 120 (Step 603) to produce signals in the operating range received at the photodetector 104 after reflection by the tissue that are within the operating range of the photodetector 104 (step 604). Light is emitted by the light source 102 at the desired wavelengths and received by the photodetector 104 upon reflectance from the tissue (step 605). The process proceeds to step 606 in which the tissue oxygen saturation and pulse oximetry values are calculated by the processor based on readings obtained from photodetector 104 (step 605). The data are then transmitted wirelessly by transceiver 130 (step 607) to remote external device 500 such as in a network, cloud, integrated vitals monitor, or mobile device such as a cell phone enabled with an app. The data received from the oximetry device are then integrated into a software application operated on microprocessor 504 and presented on a personal and/or monitoring center display 512 (step 608). A loop may be established to calculate multiple tissue oxygen saturation and pulse oximetry values by repeating steps 605, 606. In some embodiments, the device 200 includes a display unit providing tissue oxygen saturation and pulse oximetry values calculated by the microprocessor 120. In such embodiment, steps 607 and 608 are omitted.

In addition to a standard data readout, concurrent analysis using predictive software modeling in step 609 will trigger trend awareness/alarms based on clinical guardrails (step 610). For example, correlation of tissue oxygenation can be correlated with blood lactate in order to alert the clinical care teams through the monitoring center and/or integrated EHR to navigate care assessment and intervention (step 611).

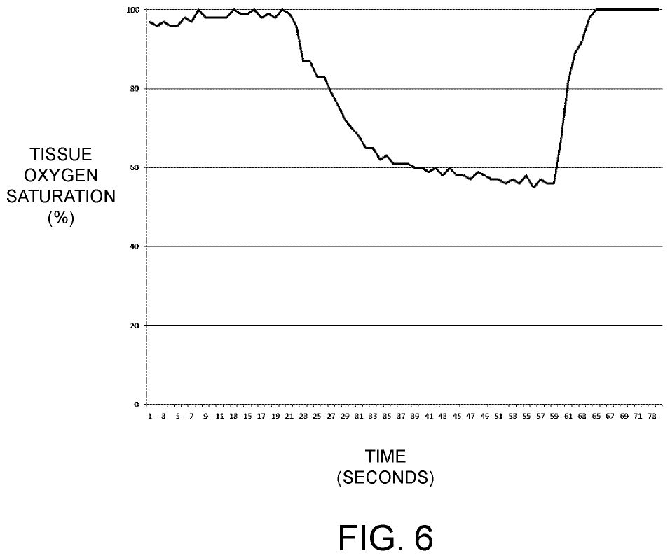

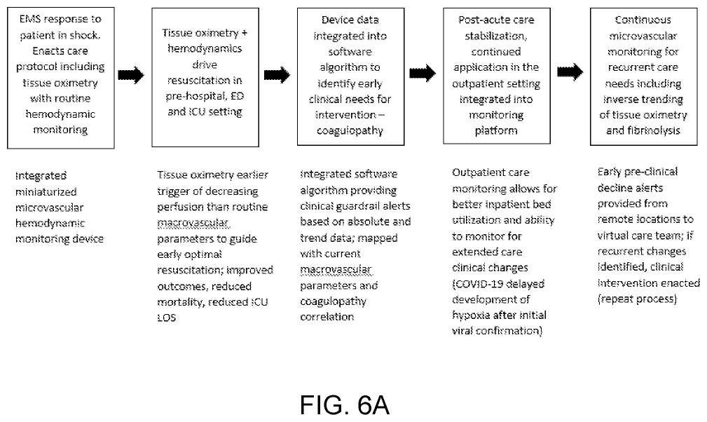

FIG. 6 is a time-course plot of tissue oxygen saturation obtained from an embodiment of the present invention. The vertical axis represents calculated tissue oxygen saturation in percent units, and the horizontal axis represents elapsed time in seconds. Sensor portion 101 was placed on the thenar eminence of the hand at time zero. Baseline measurements obtained during the first 20 seconds demonstrate initial tissue oxygen saturation readings between 95-100%. Then, a vaso-occlusive test was initiated proximal to the device (i.e., pressure applied to the tissue proximal or toward the subject's trunk, such as the forearm, to the subject's hand, thereby reducing blood perfusion) and maintained for 40 seconds. During this applied-pressure period, the measured tissue oxygen saturation values steadily declined to under 60%. When the applied pressure was removed at t=60 seconds, circulation in the tissue under the sensor was quickly restored, and a corresponding rise in tissue oxygen saturation was measured, reaching 100%. FIG. 6, therefore, demonstrates that the present invention is sensitive to changes in tissue perfusion.

FIG. 6A is a process flow diagram that integrates the content of FIG. 5 into a clinical care scenario. First responder EMS provides response to a patient in shock. EMS enacts a care protocol including tissue oximetry with routine hemodynamic monitoring. The integrated miniaturized microvascular hemodynamic monitoring device 200 described herein can be used in this stage of care. Tissue oximetry and hemodynamics drive patient resuscitation in pre-hospital, ED and ICU settings. Use of the device 200 for tissue oximetry provides an earlier trigger of decreasing perfusion than routine macrovascular parameters to guide early optimal resuscitation, improved outcomes, reduced mortality, reduced ICU length of stay (LOS).

Device data integrated into software algorithm to identify early clinical needs for intervention and/or coagulopathy. Integrated software algorithm in device 200 provide clinical guardrail alerts based on absolute and trend data; mapped with current macrovascular parameters and coagulopathy correlation.

A subsequent stage is post-acute care stabilization, utilizing continued application in the outpatient setting integrated into a monitoring platform. Outpatient care monitoring by use of device 200 allows for better inpatient bed utilization and ability to monitor for extended care clinical changes (e.g., COVID-19 delayed development of hypoxia after initial viral confirmation). Finally, continuous microvascular monitoring for recurrent care need including inverse trending of tissue oximetry and fibrinolysis. Early pre-clinical decline alerts provided by device 200 from remote locations to virtual care team; if recurrent changes identified, clinical intervention enacted.

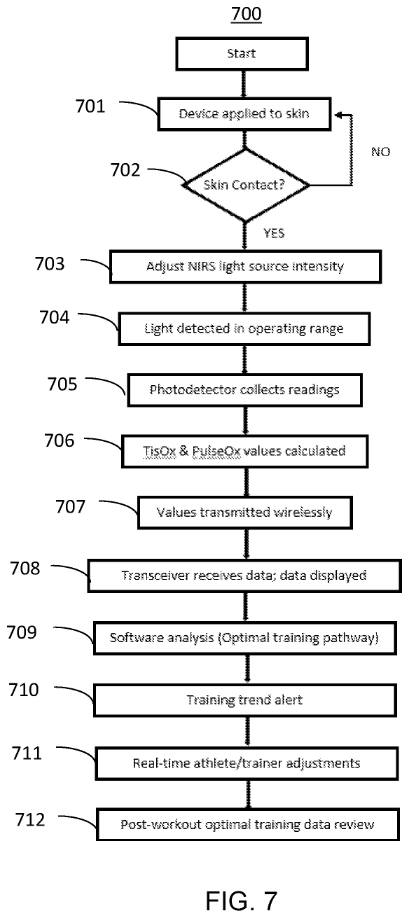

FIG. 7 is a process flow diagram in accordance with an embodiment of the present invention for athletic and fitness applications, specifically looking at the lactate threshold derived from tissue oxygen saturation versus breakpoint workload derived from lactate in accordance with an embodiment of the present invention. The process 700 begins with the application of the device to the skin in step 701. The device can be adhered to the skin with an adhesive, such as PSA previously described or held in contact with the skin by a band, sleeve or cuff. Connectivity is confirmed by the microprocessor 120, through measurements obtained from a skin contact detector 105. If skin contact has not been detected (step 702), device re-application is needed until skin contact is detected. Once skin contact is detected, the light intensities from light source 102 are automatically varied by microprocessor 120 to produce detector signals within the operating range (step 703) of photodetector 104. The process then proceeds to step 703 in which the tissue oxygen saturation and pulse oximetry values are calculated by the processor (step 706) based on readings obtained from photodetector 104 (step 705). The data are then transmitted wirelessly to a transceiver on a remote device 500 such as a smart phone, smart watch, smart wristband, etc. (step 707). The data obtained are then integrated into a software application and presented on a personal display 512 such as a smart watch or smart phone (step 708). In embodiments, the sensor unit, processor, associated software and display may be contained in a single unitary device such as a smart health watch, which may further comprise other functionality in addition to the tissue oximetry function described herein. For example, the tissue oximetry could be monitored continuously as part of a health monitoring system that includes heartrate, temperature, blood pressure, etc. The health monitoring system could include tracking trend-related parameters such as monitoring heart rate variability related to body stress.

The software modeling related to the tissue oximetry monitoring transforms the oximetry data into a user-friendly format such as a colored display value related to the current lactate threshold (step 708) as discussed further below. Training optimization guardrails will be utilized through the software to provide numerical and sound alert guidance during fitness episodes (step 709). For example, correlation of tissue oxygenation can be correlated with blood lactate in order to provide alerts regarding aerobic and anaerobic training states (step 710). In embodiments, a wearable device may be configured to allow a user to make real-time training adjustments (step 711) and post-workout optimal training reviews (step 712).

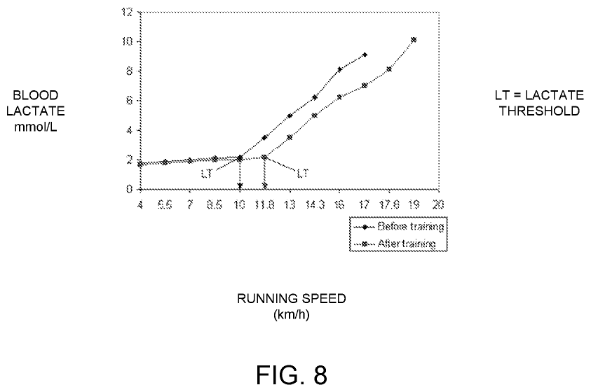

FIG. 8 is a plot of blood lactate as a function of running speed before and after physical training in accordance with an embodiment of the present invention. In fitness training, runners may be placed on a bicycle ergometer where they are presented with increasing work levels in stages. As the work level increases, for example, at increasing running speed, a point is reached in which the tissue oxygen saturation begins to drop below an established baseline. This point represents the “breakpoint” beyond which the muscle becomes increasingly hypoxic and transitions from aerobic metabolism to anaerobic metabolism, known as the lactate threshold (LT). Anaerobic metabolism results in increased muscle discomfort and decreased efficiency during continued exertion. As shown in FIG. 8, an indicator of anaerobic metabolism is increasing blood lactate levels.

Studies show that endurance athletes achieve the highest performance when they reach but do not exceed their LT during optimal training progressions. Over time, these athletes can increase their LT not just related to peak performance but also duration. In the example illustrated in the plot shown in FIG. 8, training improves the LT from 10 km/h to 11.8 km/h. For an ordinary person interested in endurance-related fitness, it would therefore be useful to be alerted in a non-invasive manner when their muscles are becoming hypoxic during exercise so that they can adjust their level of exertion to match their threshold.

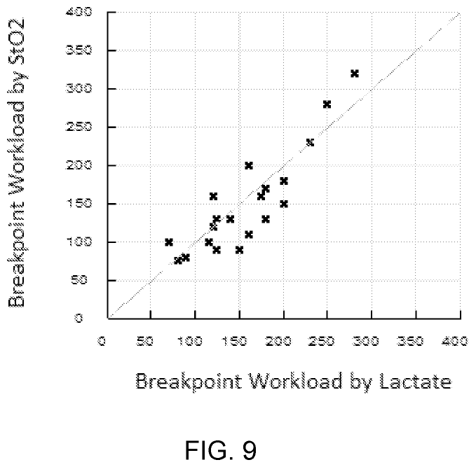

FIG. 9 is a plot of breakpoint workload derived from tissue oxygen saturation versus breakpoint workload derived from lactate in accordance with an embodiment of the present invention. As workload increases, there is an inflection point where metabolic demand is greater than tissue oxygen supply—triggering decreased tissue oxygenation and a shift to anaerobic metabolism and therefore lactate elevation. This plot was obtained from the literature and demonstrates that the breakpoint workload measured by blood lactate measurements correlates with the breakpoint workload obtained by tissue oxygen saturation measurements. As shown in FIG. 9, breakpoint workload measured by StO₂ percentage exhibits a generally 1:1 correlation with breakpoint workload measured by lactate levels.

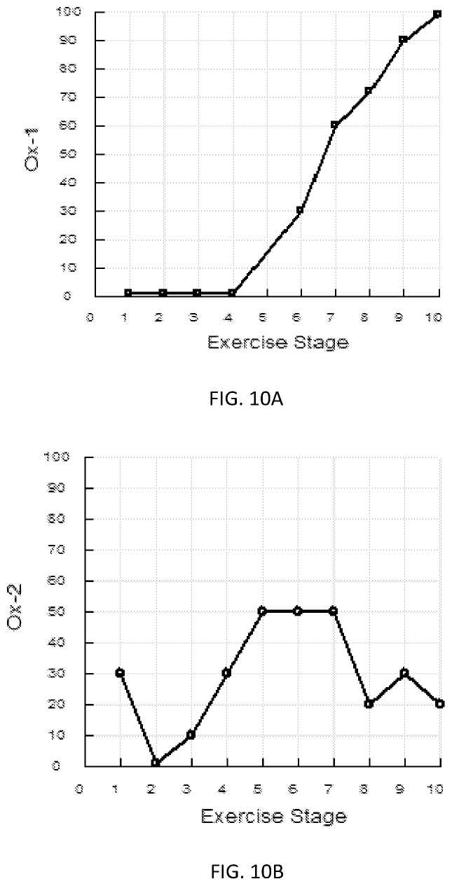

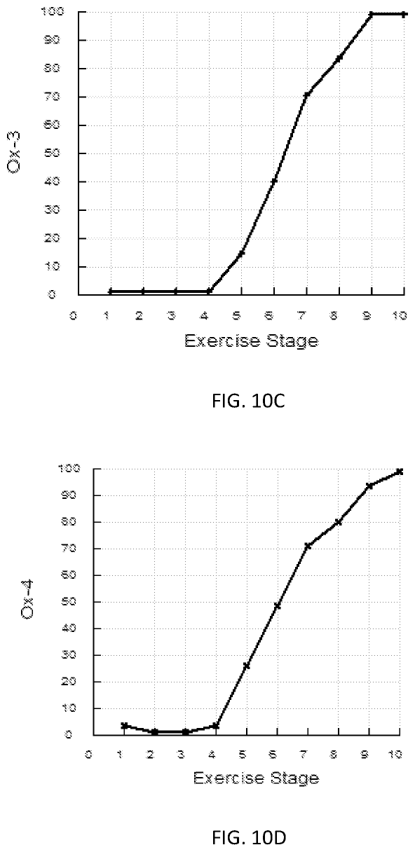

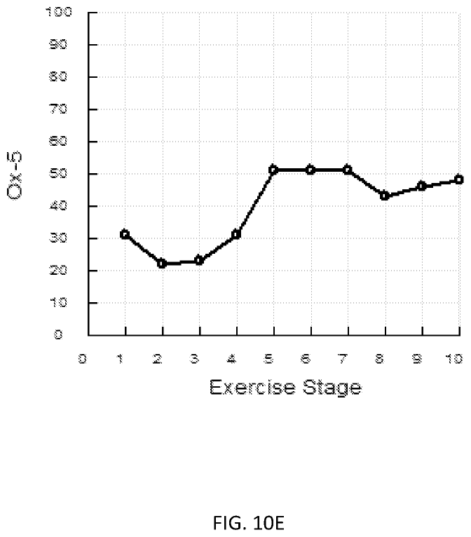

FIGS. 10A-E and 11A-E show aspects of an embodiment that provides an easy-to-use consumer-friendly index for exercise intensity level based on non-invasive tissue oxygen saturation measurements rather than invasive blood lactate measurements. Let StO₂ be the current value of tissue oxygen saturation provided by a tissue oximeter in the unit of percentage (%) and StO_{2|At rest} be the StO₂ reading at rest before exercise, also called baseline for StO₂. Then we define the following five parameters as candidates for exercise indices based on StO₂. All these exercise indices are unitless and range from 0 to 100, as follows:

Exercise Index Ox−1 (or “Index for Muscle Oxygen Level”)

Ox−1=100−StO₂. (1)

Exercise Index Ox−2 (or “Index for Muscle Oxygen Drop Rate”)

Ox−2=[Drop Rate of StO₂1×5. (2)

Here the unit of StO₂ drop rate is percentage per hour. We apply the multiplying factor 5 because StO₂ drop rates≥20%/hour is observed when blood supply is interrupted.

Exercise Index Ox−3 (or “Index for Oxygen Ratio,” or “Anaerobic Index 1”)

Ox−3=(StO_{2|At rest}/StO₂−1)×100. (3)

Exercise Index Ox−4 (or “Index for Oxygen Difference,” or “Anaerobic Index 2”)

Ox−4=StO_{2|At rest}−StO₂. (4)

Exercise Index Ox−5 (or “Combined Exercise Index”)

Ox−5=Maximum of Indices Ox−1.Ox−2.Ox−3, and Ox−4. (5)

FIGS. 10A. 10B. 10C. 10D, and 10E are plots of exercise index Ox−1. Ox−2, Ox−3, Ox−4, and Ox−5, respectively, versus the exercise stage in accordance with an embodiment of the present invention. The values of these five exercise indices are graphically shown at different stages or intervals of exercise at a level above a maintenance threshold, that is, at a sustained performance level. In the calculation of the StO₂ drop-rate, the time duration employed for each stage is 30 minutes. Statistical parameters for the time series of the indices are listed in Table 1. All exercise indices at rest before exercise are of a small value close to zero. As the oximeter-user exercises, the muscle StO₂ decreases, and all the exercise indices and the lactate value rise as a trend. As shown in FIG. 9, work as represented by oxygen levels and blood lactate are generally correlated. Therefore, the exercise indices are correlated to the lactate value, and both remain near their baselines until the muscle becomes hypoxic, transitioning from aerobic metabolism to anaerobic metabolism. Also, both parameters rise as a trend when exercise intensity increases.

TABLE 1
Comparison between the five exercise indices.

| Exercise Index | Baseline required | R² | p-value |
|---|---|---|---|
| Ox-1 | No | 0.92 | 0.22 |
| Ox-2 | No | 0.61 | 0.21 |
| Ox-3 | Yes | 0.92 | 0.20 |
| Ox-4 | Yes | 0.92 | 0.13 |
| Ox-5 | Yes | 0.98 | 0.22 |

Consider the following linear calibration for the “five exercise indices.”

Ox−i=Ox−i X k+b,i=1.2,3,4,5. (6)

Here k and b are linear calibration factors. When the calibration factors are listed in Table 2 below, the calibrated exercise indices are graphically shown in FIGS. 10A through 10E.

TABLE 2
Factors for linear calibration.

| Exercise Index | k | b |
|---|---|---|
| Ox-1 | 6 | −180 |
| Ox-2 | 1 | 0 |
| Ox-3 | 3 | −20 |
| Ox-4 | 4.5 | −10 |
| Ox-5 | 1 | 0 |

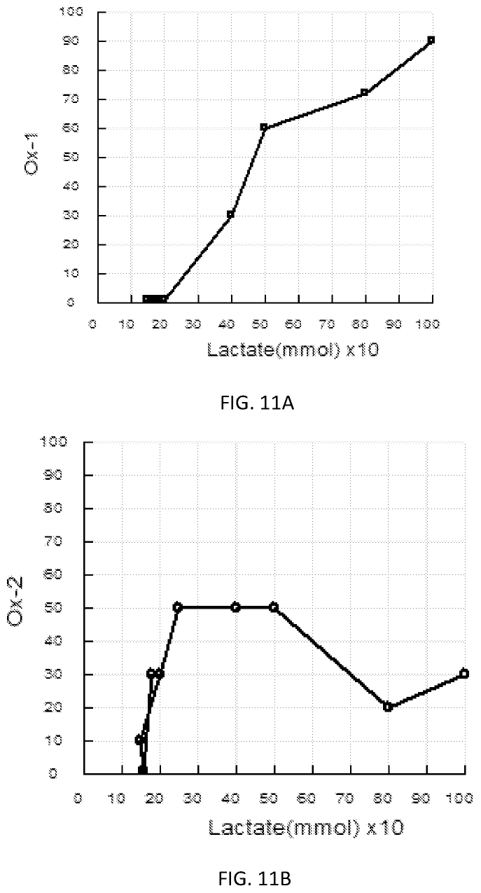

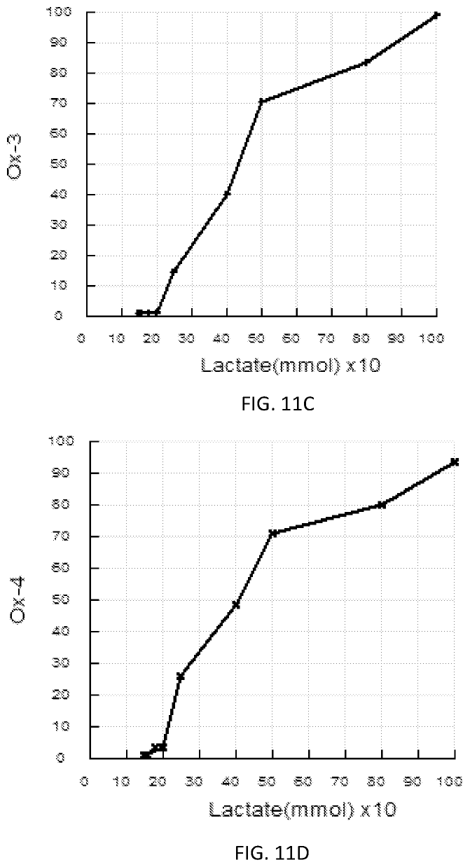

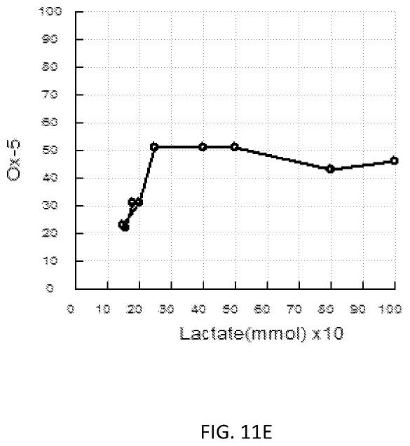

FIGS. 11A. 11B. 11C. 11D, and 11E are plots of exercise index Ox−1. Ox−2, Ox−3, Ox−4, and Ox−5 respectively versus lactate in accordance with an embodiment of the present invention. As can be seen by inspection of these figures. Ox−1, Ox−3 and Ox−4 show a generally 1:1 correlation with blood lactate levels throughout the stages. Ox−2 and Ox−5 correlate with lactate levels until about 25 mmol×10, after which they plateau and then drop at lactate levels at about 50 mmol×10.

In an embodiment of the present invention, an exercise index value can be displayed numerically and/or in the form of an easy-to-understand red, yellow, and green light indicators 520/522/524. The tissue oximetry indicators for the exercise index values are customizable to an individual and are adaptive because the anaerobic trigger changes with increased training. For example, the lactate threshold for an individual changes as training increases as well as current physiologic state of health, rest and recovery, reflecting the individual's improved ability to perform, as shown in FIG. 8. The green light 524 indicates the intensity of exercise is in the aerobic range, and exercise may continue. The yellow light 522 indicates a transition from an aerobic to an anaerobic state, and red light 520 means reaching the anaerobic range. These indicators illustrate the current state and can be used to refine the training regimen based on each individual's desired outcomes.

The foregoing disclosure provides illustration and description but is not intended to be exhaustive or to limit the embodiments or implementations to the precise form disclosed. Variations to the disclosed embodiments and/or implementations may be understood and effected by those skilled in the art in practicing the claimed disclosure, from a study of the drawings, the disclosure, and the appended claims. Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A portable tissue oximeter, comprising:
a wearable sensor unit defining a contact surface comprising
a skin contact detector comprising at least one electrode configured to provide a detection signal when the contact surface is in contact with the portion of the subject's skin to be analyzed;
at least one tunable light source arranged to provide a first beam of light towards the portion of the subject's skin to be analyzed at a first wavelength and a second beam of light at a second wavelength;
a photodetector arranged to receive the first beam of light and the second beam of light that are reflected from the portion of the subject's skin to be analyzed;
a sensor unit wireless transceiver; and
a sensor unit microprocessor and a computer-readable storage medium storing instructions that when executed by the sensor unit microprocessor cause the sensor unit microprocessor to
emit the first beam of light from the light source upon receipt of the detection signal;
measure the output of the photodetector receiving the reflection of the first light beam from the tissue;
emit the second beam of light from the light source;
measure the output of the photodetector receiving the reflection of the second light beam from the tissue;
compute oxygen saturation values based on photodetector measurements; and
transfer a signal of the oxygen saturation values from the sensor unit wireless transceiver;
an external device comprising
an external device wireless transceiver;
an external device microprocessor and a computer-readable storage medium storing instructions that when executed by the external device microprocessor cause the external device microprocessor to receive oxygen saturation values form the sensor unit transceiver and determine outputs to a user, and
a user interface to provide user notifications of oxygen saturation and alerts.

2. The tissue oximeter of claim 1, wherein the at least one tunable light source emits the first beam of light at one or more wavelengths.

3. The tissue oximeter of claim 1, wherein the at least one tunable light source emits the second beam of light at a wavelength of 660 nm.

4. The tissue oximeter of claim 1, wherein the skin contact detector measures the capacitance of a region of the skin under the detector.

5. The tissue oximeter of claim 1, wherein the portion of the subject's skin to be analyzed is one of the thenar eminence, the deltoid, the hamstring, the quadriceps, the adductor muscle of the thigh, the gastrocnemius muscle of the calf, and the temporal/lateral forehead region of the subject.

6. The tissue oximeter of claim 1 wherein the wearable sensor unit further comprising a fixation system configured to fully contain the wearable sensor unit and secure it to the skin.

7. The tissue oximeter of claim 6, wherein the fixation system comprises a pressure-sensitive adhesive or a strap.

8. The tissue oximeter of claim 6, wherein the fixation system comprises a biocompatible film transparent to wavelengths emitted by the at least one tunable light source and the photodetector to separate the at least one tunable light source, the photodetector, and skin contact detector from the skin.

9. The tissue oximeter of claim 6, wherein the fixation system comprises:
a first compartment to house the at least one tunable light source, the photodetector, and the skin contact sensor, wherein the first compartment is oriented to align with the portion of the subject's skin to be analyzed;
a second compartment to house the sensor unit microprocessor, and
a third compartment to house the sensor unit wireless transceiver.

10. The tissue oximeter of claim 6, wherein the fixation system comprises:
a first compartment to house the at least one tunable light source, the photodetector, and the skin contact sensor, wherein the first compartment is oriented to align with the portion of the subject's skin to a be analyzed; and
a second compartment to house the sensor unit microprocessor and the sensor unit wireless transceiver.

11. The tissue oximeter of claim 6, wherein the fixation system comprises:
a single compartment to house the at least one tunable light source, the photodetector, the skin contact sensor, the sensor unit microprocessor and the sensor unit wireless transceiver.

12. The tissue oximeter of claim 1, wherein the wearable sensor unit defines a volume of less than 20 ml.

13. The tissue oximeter of claim 1, wherein the wearable sensor unit defines a volume of less than 6 ml.

14. The tissue oximeter of claim 1, wherein the user interface comprises a display screen to display the oxygen saturation values, alerts, and an indication of skin contact.

15. The tissue oximeter of claim 1, wherein the external device is a mobile phone.

16. The tissue oximeter of claim 1, wherein the sensor unit transceiver and external unit transceiver communicate via cellular, Bluetooth, NFC or WiFi.

17. The tissue oximeter of claim 1, wherein the external device comprises a DC power supply.

18. The tissue oximeter of claim 1, wherein the wearable sensor unit comprises a DC power supply.

19. The tissue oximeter of claim 1 wherein the sensor unit microprocessor or the external unit microprocessor is configured to correlate blood lactate measurements with the tissue oxygen saturation measurements.

20. The tissue oximeter of claim 19, wherein the sensor unit microprocessor or the external unit microprocessor is configured to determine whether the subject is in an aerobic state, in a transition from an aerobic to an anaerobic state, and in an anaerobic state based on the correlation between blood lactate measurements and the tissue oxygen saturation measurements.

21. The tissue oximeter of claim 20, wherein the user interface provides an alert regarding the state of the subject in an aerobic state, in a transition from an aerobic to an anaerobic state, and in an anaerobic state.

22. A wearable tissue oximeter, comprising:
a sensor unit defining a contact surface comprising
a skin contact detector comprising at least one electrode configured to provide a detection signal when the contact surface is in contact with the portion of the subject's skin to be analyzed;
at least one tunable light source arranged to provide a first beam of light towards the portion of the subject's skin to be analyzed at a first wavelength and a second beam of light at a second wavelength;
a photodetector arranged to receive the first beam of light and the second beam of light that are reflected from the portion of the subject's skin to be analyzed;
a microprocessor and a computer-readable storage medium storing instructions that when executed by the microprocessor cause the microprocessor to
emit the first beam of light from the light source upon receipt of the detection signal;
measure the output of the photodetector receiving the reflection of the first light beam from the tissue;
emit the second beam of light from the light source;
measure the output of the photodetector receiving the reflection of the second light beam from the tissue;
compute oxygen saturation values based on photodetector measurements; and
a user interface to provide user notifications of oxygen saturation and alerts.

23. The wearable tissue oximeter of claim 22, wherein the at least one tunable light source emits the first beam of light at one or more wavelengths.

24. The wearable tissue oximeter of claim 22, wherein the at least one tunable light source emits the second beam of light at a wavelength of 660 nm.

25. The wearable tissue oximeter of claim 22, wherein the skin contact detector measures the capacitance of a region of the skin under the detector.

26. The wearable tissue oximeter of claim 22, wherein the portion of the subject's skin to be analyzed is one of the thenar eminence, the deltoid, the hamstring, the quadriceps, the adductor muscle of the thigh, the gastrocnemius muscle of the calf, and the temporal/lateral forehead region of the subject.

27. The wearable tissue oximeter of claim 22, wherein the sensor unit further comprising a fixation system configured to fully contain the sensor unit and secure it to the skin.

28. The wearable tissue oximeter of claim 27, wherein the fixation system comprising a pressure-sensitive adhesive or a strap.

29. The wearable tissue oximeter of claim 27, wherein the fixation system comprises a biocompatible film transparent to wavelengths emitted by the at least one tunable light source and the photodetector to separate the at least one tunable light source, the photodetector, and skin contact detector from the skin.

30. The wearable tissue oximeter of claim 27, wherein the fixation system comprises:
a first compartment to house the at least one tunable light source, the photodetector, and the skin contact sensor, wherein the first compartment is oriented to align with the portion of the subject's skin to a be analyzed; and
a second compartment to house the microprocessor.

31. The wearable tissue oximeter of claim 27, wherein the fixation system comprises:
a single compartment to house the at least one tunable light source, the photodetector, the skin contact sensor, and the microprocessor.

32. The wearable tissue oximeter of claim 22, wherein the user interface comprises a display screen to display the oxygen saturation values, alerts, and an indication of skin contact.

33. The wearable tissue oximeter of claim 22, wherein the sensor unit microprocessor or the external unit microprocessor is configured to correlate blood lactate measurements with the tissue oxygen saturation measurements.

34. The wearable tissue oximeter of claim 33, wherein the sensor unit microprocessor or the external unit microprocessor is configured to determine whether the subject is in an aerobic state, in a transition from an aerobic to an anaerobic state, and in an anaerobic state based on the correlation between blood lactate measurements and the tissue oxygen saturation measurements.

35. The wearable tissue oximeter of claim 34, wherein the user interface provides an alert regarding the state of the subject in an aerobic state, in a transition from an aerobic to an anaerobic state, and in an anaerobic state.
